(19) United States
(12) Patent Application Publication
(10) Pub. No.: US 2008/0132714 A1
(43) Pub. Date: Jun. 5, 2008

(54) Manufacture of Esters Using a Multiple Catalyst Approach

(76) Inventor: D. Kip Hillshafer, Western Springs, IL (US)

Correspondence Address: MCANDREWS HELD & MALLOY, LTD, 500 WEST MADISON STREET, SUITE 3400, CHICAGO, IL, 60661, US

(21) Appl. No.: 11/629,324

(22) PCT Filed: May 9, 2005
(86) PCT No.: PCT/US05/16196

§ 371 Date: Sep. 14, 2007

Related U.S. Application Data
(60) Provisional application No. 60/580,936, filed on Jun. 18, 2004.

(51) Int. Cl. C07C 67/08; C07D 307/60

(52) U.S. Cl. 549/256; 560/77

(57) ABSTRACT

Esters are prepared from hydroxyl-containing compounds and carboxylic acids or anhydrides or lower alkyl esters thereof using a multiple catalyst approach, wherein a first catalyst such as an organotin catalyst can be present at the beginning of the reaction and a second catalyst such as an organotitanium catalyst can be added any time after the addition of the first catalyst, or when the acid value of the reaction mixture falls below a predetermined acid value. The second catalyst can be added multiple times, and a third or additional catalysts can also be used.

### RELATED APPLICATIONS

[Not Applicable]

### FIELD OF THE INVENTION

This invention generally relates to the use of a first organometallic catalyst followed by at least one other organometallic catalyst in the manufacture of an ester product. This technique, called a “multiple catalyst” approach, has the advantageous result of reducing the polymerization, reaction, esterification and/or transesterification time during the manufacture of esters, such as polyesters. Additionally, such a technique allows for the production of low acid value polyester products.

### BACKGROUND OF THE INVENTION

Catalysts are normally used in the manufacture of polyesters. Typically a single catalyst is used during a single production of the material. For example, production of phthalate esters (e.g., STEPANPOL® polyols) usually involves the esterification of phthalic anhydride and at least one glycol. Such processes may be typically catalyzed with the use of an organotitanium catalyst (e.g., “TYZOR® TBT”, available from Du Pont Chemical, Wilmington, Del.) or an organotin catalyst (e.g., “FASCAT® 4102”, available from ATOFINA, Philadelphia, Pa.).

It is well known that organotin compositions, including organotin oxides, hydroxides, alkoxides and carboxylates are effective as catalysts in the manufacture of polyester resins and polyester-containing compositions. The use of tin catalysts in the esterification of polyesters is disclosed by Caldwell in U.S. Pat. No. 2,720,507, by Dombrow, et al. in U.S. Pat. No. 3,162,616, by Allison, et al. in U.S. Pat. No. 3,345,339, by Cook in U.S. Pat. No. 3,716,523 and by Jackson, Jr., et al. in U.S. Pat. No. 4,554,334. The use of organotin catalysts decreases the time required to complete esterification or transesterification of polyester compositions and to effectuate a complete reaction.

U.S. Pat. No. 4,970,288 (Larkin, et al.) describes the use of non-toxic organotin esterification catalysts in the production of polyester and polyester-containing compositions. U.S. Pat. No. 5,166,310 (Rooney) also describes a process for the preparation of polyesters in the presence of a combination of tin catalysts only.

U.S. Pat. No. 4,393,191 (East) describes a process of direct polymerization of aromatic hydroxyl acids which is conducted in the presence of a group IV or V metallic catalyst. The catalyst described is a salt, oxide or organometallic derivative of either antimony, titanium, tin or germanium, with tin compounds being the most preferred for reasons of catalyst activity.

U.S. Pat. No. 4,837,245 (Streu, et. al.) describes a method to prepare a polyester polyol through the polycondensation of organic polycarboxylic acids with multivalent alcohols in the presence of from 0.002 to 5 weight percent, based on the weight of the mixture composed of polycarboxylic acids and multivalent alcohols, of at least one titanium and/or tin compound, preferably an organic titanic acid ester having the structure Ti(OR)₄ in which R stands for a linear, branched or cyclic alkyl radical having from 1 to 6 carbon atoms.

Polyesters are formed by the condensation of a dibasic or polybasic acid and a dihydric or polyhydric alcohol to form a series of ester linkages. For example, aromatic polyester polyols, especially phthalate polyester polyols, are produced by esterifying aromatic polycarboxylic acids with polyols. Optionally, a tri-functional or polyfunctional alcohol or acid functional branching agent may be used to enhance the properties of the polyester or polyester-containing material formed from the polyester. Optionally, monofunctional monomers such as benzoic acid or stearic acid can also be used to control molecular weight.

Esters can also be prepared by transesterification reactions. When using the ester-interchange method, the long chain in the polyester is built up by a series of ester interchange reactions wherein the glycol displaces an ester to form the glycolester. Included are the reactions between two esters to yield two new esters, as well as transesterification reactions where the components of the esters involved are polyhydroxy alcohols and polybasic acids.

A polyester can typically be prepared by heating the condensing mixture at temperatures of at least about 160° C. up to about 250° C. or higher in order to maintain the fluid state. The reaction can be performed above atmospheric pressure, up to about 20 psig (14062 kg/m²) or higher, at atmospheric pressure or under vacuum to about 15 mm Hg or lower. The esterification reaction can be conducted in the presence of a suitable solvent such as toluene or xylene, and the like. Nitrogen, argon, helium or any other suitable gas may be introduced into the reactor to keep air out of the reactor or to facilitate the removal of water, low-boiling alcohol, mixtures thereof or the like. The polyesters can also be thinned in a suitable reactive monomer such as styrene or divinyl benzene, or mixtures thereof and the like.

However, it would be desirable in the polyester field to have available a process for the preparation of polyesters which would result in less manufacturing time (i.e., polymerization, reaction, esterification, and/or transesterification processing time) as well as lower acid value polyester products.

### BRIEF SUMMARY OF THE INVENTION

The presently described technology pertains to an improved process for the preparation of esters, preferably low acid value polyesters, by reacting a hydroxyl-containing compound with a carboxyl-containing compound, anhydride or lower alkyl ester thereof using a multiple catalyst approach.

It has now surprisingly and unexpectedly been found that by using a first catalyst, preferably an organotin catalyst, followed sequentially by a second catalyst, preferably an organotitanium catalyst, in the manufacture of an ester results in an enhanced and more efficient manufacturing process. It can also produce lower final acid value polyester products as compared to conventional manufacturing processes utilizing only an organotin or organotitanium catalyst. The second catalyst can also be added at the same time as or any time after the first catalyst is added. Further, the second catalyst can be added multiple times or a third or additional catalysts can be utilized in accordance with the presently described technology.

Furthermore, it has also been found that if an organotin catalyst is present at the beginning of the reaction and is followed by at least one addition of an organotitanium catalyst at one particular point in the reaction (as set by a predetermined acid value), then the multiple catalyst approach provides improved reaction kinetics.

### BRIEF DESCRIPTION OF SEVERAL VIEWS OF THE DRAWINGS

[Not Applicable]

### DETAILED DESCRIPTION OF THE INVENTION

The multiple catalyst approach of the presently described technology is applied by the use of two or more catalysts. Essentially all other variables used in the conventional manufacture of esters, such as polyester polyols or polyester resins, including temperature, agitation, nitrogen sparging, vacuum evaporation and the like, can remain unchanged.

The presently described technology, involving two catalyst additions, can be referred to as a “dual catalyst” approach. However, it should be understood by a person of ordinary skill in the art that more than two catalysts and/or more than two additions of the catalysts can be used in the reaction of the presently described technology. It should also be understood that other orders of addition of the two or more catalysts beyond those explicitly listed in this specification are also contemplated.

In one embodiment of the presently described technology, a first catalyst, preferably an organotin catalyst, can be present at the beginning of the manufacturing reaction, and at any time after the addition of the first catalyst, a second catalyst, preferably an organotitanium catalyst, can be added to the reaction system. In another embodiment, two or more catalysts can be added together or at about the same time to the reaction system.

In a further embodiment of the presently described technology, the first catalyst, preferably an organotin catalyst, can be present at the beginning of the manufacturing reaction, but the second catalyst, preferably an organotitanium catalyst, can be added to the reaction system at a later point of the reaction, as measured by a predetermined acid value.

In yet another embodiment of the present technology, the first catalyst, preferably an organotin catalyst, can be present at the beginning of the manufacturing reaction, and the second catalyst, preferably an organotitanium catalyst, can be added multiple times to the reaction system at different points of the reaction anytime after the addition of the first catalyst; or the second catalyst and one or more additional catalysts can be added to the reaction system at different points of the reaction anytime after the addition of the first catalyst. In other words, after both the first and second catalysts have been added once to the reaction system, an additional charge of another effective amount of the second catalyst or a third catalyst (or other additional catalysts) can be added to the system, and this additional charging step can be performed more than once with the same or a different catalyst. When the second or additional catalysts are added at one particular point or multiple points during the reaction, such point(s) can be, for example, when the acid value of the reaction mixture falls below 100, alternatively falls below 50, alternatively falls below 30, and alternatively falls below 10.

“Acid value or number” is a measure of free acid content of a substance. It is expressed as the number of milligrams (mg) of potassium hydroxide (KOH) neutralized by the free acid present in one gram (g) of the substance (unit: “mg KOH/g”). This value is sometimes used in connection with the end-group method of determining the molecular weight of polyesters. It is also used in evaluating plasticizers, in which acid values should be as low as possible.

Similarly “hydroxyl number” is defined as the number of milligrams (mg) of potassium hydroxide (KOH) required for the complete neutralization of the hydrolysis product of a fully acetylated derivative prepared from one gram (g) of a polyol or a mixture of polyols (unit: “mg KOF/g”). The term “hydroxyl number” is also defined by the equation:

OHV=56.1×1000×F/M.W.

wherein;
OHV is the hydroxyl number (of the polyol or polyol blend), F is the average functionality (i.e., the average number of active hydroxyl groups per molecule of the polyol or polyol blend), and M.W. is the average molecular weight of the polyol or polyol blend.

The multiple catalyst approach of the presently described technology can be used for all types of esterification reactions between hydroxyl groups and carboxylic acids, carboxylates and/or anhydrides. When a tin/titanium (Sn/Ti) dual catalyst approach is used, the esterification reaction can be generally represented by the following scheme:

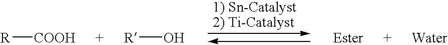

The process of the presently described technology can be conducted at temperatures usually employed in the preparation of polyesters of from about 150° C. to about 290° C., preferably from about 170° C. to about 250° C., and more preferably from about 180° C. to about 240° C. The esterification or transesterification reaction can be conducted at pressures, for example, from about 700 mm Hg to about 1,500 mm Hg. The reaction temperature and pressure can be balanced such that the water or low-boiling alcohol of reaction is removed as quickly as possible while not distilling the low-boiling reactants, generally glycols, from the reaction. It is generally advantageous to finish the reaction at reduced pressure, generally below about 100 mm Hg, preferably below about 50 mm Hg, and more preferably below about 10 mm Hg.

The reaction is conducted for a sufficient time to bring the reaction to the desired degree of completion. It should be understood by one skilled in the art that the time required to achieve the desired degree of reaction depends upon many factors, such as heating and cooling capacity, reaction vessel size, particular reactants and catalyst utilized, and the like. Quite surprisingly and unexpectedly, however, the multiple catalyst approach of the presently described technology (when used in the laboratory setting), has had the advantageous result of reducing the time for polymerization, reaction, esterification and/or transesterification of a 415 molecular weight dipropylene glycol-phthalate from about 28 hours in that setting (with exclusive use of the FASCAT® 4102 catalyst, available from ATOFINA, Philadelphia, PA) or more (with the exclusive use of the TYZOR® TBT catalyst, available from Du Pont Chemical, Wilmington, Del.) to about 13 hours when roughly equal amounts of the tin- and titanium-based catalysts were used. All other reaction conditions, including temperature, agitation speed, nitrogen sparge rate and the like were essentially the same for these comparisons.

In accordance with one embodiment of the presently described technology, an organotin catalyst (e.g., FASCAT® 4102) can typically be charged at a level of about 250 ppm immediately after all phthalate and glycols have been added to a reactor. The reaction can typically be heated to about 200° C. to about 215° C. with agitation, nitrogen sparge and/or a vacuum profile if desired and allowed to progress until the reaction acid value falls below, for example, about 30 mg KOH/g, at which point 250 ppm of an organotitanium catalyst (e.g., TYZOR® TBT) can be added. After such an addition, all usual polyester manufacturing reaction conditions can be resumed and maintained throughout the course of the product synthesis.

It should be understood by those skilled in the art that any carboxyl-containing compound (i.e., organic acid such as R-COOH) or derivatives thereof can be used in the reaction of the presently described technology. Aromatic, cycloaliphatic and aliphatic dicarboxylic acids having from about 2 to about 20 carbon atoms are preferably used as the organic polycarboxylic acid. Examples of dicarboxyl-containing compounds, anhydrides, or lower alkyl (C₁-C₄) esters include, but are not limited to phthalic acid, isophthalic acid, terephthalic acid, succinic acid, glutaric acid, adipic acid, azelaic acid, oxalic acid, sebacic acid, fumaric acid, suberic acid, hexahydrophthaic acid, succinic anhydride, phthalic anhydride, phthalates, adipates, isophthalates, terephthalates, maleic anhydride, promellitic dianhydride, chlorendic anhydride, 5-sodiosufoisophthalic acid, trimelletic anhydride, and mixtures thereof.

It should be understood by those skilled in the art that any hydroxyl-containing compounds (e.g., alcohols such as R′-OH) can be used in the reaction of the presently described technology. Dihydroxyl-containing compounds (i.e., diols) which can be employed herein, include, for example, aliphatic, cycloaliphthalic or aromatic diols which can be either saturated or unsaturated. Such diols can have from about 2 to about 20, preferably from about 2 to 12 carbon atoms, more preferably from about 2 to about 6 carbon atoms per molecule. Examples of dihydroxyl-containing compounds include, but are not limited to diethylene glycol, triethylene glycol, tetraethylene glycol, dipropylene glycol, tripropylene glycol, dibutylene glycol, tributylene glycol and tetrabutylene glycol, neopentyl glycol, methyl propyl diol, ethylene glycol, propylene glycol, 1,4-butanediol, 1,3-butanediol, 1,2-butanediol, 1,5-pentanediol, 1,6-hexanediol, heptanediol, nonanediol, decanediol, 2,2,4-trimethyl-1,3-pentanediol, cyclohexanedimethanol, 2-methyl-1,3-propanediol, polyoxyalkylene-diols having molecular weights from about 96 to about 600, more preferably from about 96 to about 300 based on ethylene oxide, 1,2-propylene oxide, tetrahydrofuran, and mixtures thereof. Other suitable hydroxyl-containing compounds include but are not limited to ε-caprolactone, glycerin, sorbitol, trimethylolpropane, sucrose, propylene oxide and/or ethylene oxide adducts of sucrose, trimethylolpropane or glycerine, castor oil, tris-2-hydroxyethyl isocyanate (THEIC), polypropylene glycol, polyethylene glycol, and mixtures thereof

It should also be understood by those skilled in the art that any organotin and organotitinium catalysts or other organometallic catalyst suitable for esterification reactions can be used in the multiple catalyst approach of the presently described technology.

Suitable organotin salts of a carboxylic acid which can be employed in the presently described technology include, for example, those represented by the following formulas: R—Sn(O₂CR′), R₂Sn(O₂R′)₂, R₂Sn(O₂CR′)(OCR′), R—Sn(O₂CR′)₃ or R—Sn(O₂CR′)₂Y; wherein each R can be an alkyl group having from about 1 to about 20 carbon atoms, preferably from about 1 to about 12 carbon atoms, more preferably from about 1 to about 8 carbon atoms, or an aryl, alkaryl or cycloalkyl group having from about 6 to about 14 carbon atoms; each R′ can be an alkyl group having from about 1 to about 20 carbon atoms, preferably from about 1 to about 12 carbon atoms, more preferably from about 1 to about 8 carbon atoms, or an aryl, alkaryl or cycloalkyl group having from about 6 to about 14 carbon atoms. When there are more than one R (or R′) in the same molecule, each R (or R′) can be the same or different group. The R groups can be saturated or unsaturated and can also be substituted or unsubstituted with such substituent groups as alkyl, aryl or cycloalkyl groups having from about 1 to about 20 carbon atoms, a halogen, preferably chlorine or bromine, —NO₂, and the like. Y is a halogen, preferably chlorine or bromine.

Such organotin salts of a carboxylic acid can include, but are not limited to stannous acetate, stannous laurate, stannous octoate, stannous oleate, stannous oxalate, dibenzyltin diacetate, dibenzyltin distearate, dibutylmethoxytin acetate, dibutyltin diacetate, dibutyltin dilaurate, dibutyltin maleate, dilauryltin diacetate, dioctyltin diacetate, dioctyltin dilaurate, diphenyltin diacetate, methyltin trilaurate, methyltin tris(2-ethylhexoate), butyltin triacetate, butyltin trilaurate, butyltin tris(2-ethylhexoate), or any combination thereof. Particularly suitable such organotin salts of carboxylic acids include, for example, dibutyltin diacetate, dibutyltin dilaurate, stannous octoate, dioctyltin dilaurate or any combination thereof.

Suitable organotin oxides which can be employed in the presently described technology include, for example, those represented by the formula R₂SnO wherein each R can be the same or different and can be an alkyl group having from about 1 to about 20 carbon atoms, preferably from about 1 to about 12 carbon atoms, more preferably from about 1 to about 8 carbon atoms, or an aryl, alkaryl or cycloalkyl group having from about 6 to about 14 carbon atoms. The R groups can be saturated or unsaturated and can also be substituted or unsubstituted with such substituent groups as alkyl, aryl or cycloalkyl groups having from about 1 to about 20 carbon atoms, a halogen, preferably chlorine or bromine, —NO₂, and the like. Such organotin oxides include, but are not limited to bis(carbomethoxyethyl) tin oxide, diallyltin oxide, dibenzyltin oxide, dibutyltin oxide, dicyclohexyltin oxide, dilauryltin oxide, dimethyltin oxide, di-1-naphthyltin oxide, dioctyltin oxide, diphenyltin oxide, divinyltin oxide, or any combination thereof. Particularly suitable organotin oxides include, for example, dibutyltin oxide, dimethyltin oxide or any combination thereof.

Suitable organostannoic acids which can be employed herein include, for example, those represented by the formula R—SnOOH or their corresponding anhydrides represented by the formula (R—SnO)₂O wherein each R can be an alkyl group having from about 1 to about 20 carbon atoms, preferably from about 1 to about 12 carbon atoms, more preferably from about 1 to about 8 carbon atoms, or an aryl, alkaryl or cycloalkyl group having from about 6 to about 14 carbon atoms. When there are more than one R in the same molecule, each R can be the same or different group. The R groups can be saturated or unsaturated and can also be substituted or unsubstituted with such substituent groups as alkyl, aryl or cycloalkyl groups having from about 1 to about 20 carbon atoms, a halogen, preferably chlorine or bromine, —NO₂, and the like. Such organostannoic acids or anhydrides thereof include, but are not limited to phenylstannoic acid, chlorobenzylstannoic acid, 1-dodecyl-stannoic acid, methylstannoic acid, 1-naphthylstannoic acid, butylstannoic acid, octylstannoic acid, anhydrides of such acids, or any combination thereof Particularly suitable organostannoic acids or anhydrides include, for example, butylstannoic acid, methylstannoic acid or any combination thereof.

Suitable titanium compounds that can be utilized in the presently described technology include, for example, ortho-titanates having the formula Ti(OR)₄ in which R is a cyclic, branched or linear alkyl radical having from about 1 to about 6 carbon atoms, more preferably from about 1 to about 4 carbon atoms. Typical orthotitanates include, for example: tetramethyl-, tetraethyl-, tetra-n-butyl-, tetraisobutyl-, tetra-sec-butyl-, tetra-tert-butyl, tetraisopropyl-, tetraphenyl-, tetra-n-pentyl-tetra-n-pentenyl- and tetra-n-hexyl-titanate. Other examples also include, but are not limited to titanium alkoxides, and dicyclopentadienyldiphenyl titanium. Tetra-n-butyl-titanate is an example of a preferred organic titanium esterification catalyst of the presently described technology.

Examples of other suitable organometallic catalysts include, but are not limited to lead acetate, lead octoate, cobalt acetate, magnesium acetate and calcium acetate.

The amount of catalyst employed is a catalytically effective amount, which is an amount sufficient to increase the rate of polymerization, which can be measured by, for example, conventional means such as the inherent viscosity, acid value, or hydroxyl value of the resultant polyester. For example, the organotin catalyst of the present technology can be employed as a first catalyst in an amount of from about 5 ppm to about 1500 ppm, preferably from about 10 ppm to about 1000 ppm, more preferably from about 50 ppm to about 700 ppm, even more preferably from about 100 ppm to about 500 ppm based on the total initial reactant charge weight. The organotitanium catalyst of the present technology, for example, can be employed as a second catalyst in an amount of from about 5 ppm to about 1500 ppm, preferably from about 10 ppm to about 1000 ppm, more preferably from about 50 ppm to about 700 ppm, even more preferably from about 100 ppm to about 500 ppm based on the total initial reactant charge weight. However, it should be understood by one of ordinary skill in the art that the ranges of the organotin or organotitanium (or other organometallic) catalysts utilized can be increased or decreased as deemed appropriate and effective, and get to achieve the goal of the presently described technology.

The presently described technology and its advantages will be better understood by reference to the following examples. These examples are provided to describe specific embodiments of the present technology. By providing these specific examples, the inventor does not limit the scope and spirit of the present technology. It will be understood by those skilled in the art that the full scope of the presently described technology encompasses the subject matter defined by the claims appending this specification, and any equivalents of those claims.

### EXAMPLES

### Comparative Example 1

### Preparation Of Polyester Polyols Using Laboratory Batches

Approximately 888 grams of phthalic anhydride (Koppers, Pittsburgh, Pa.) were charged into each of two separate, suitable four-finger roundbottom flasks, followed by approximately 1635 g of dipropylene glycol (Dow Chemical, Midland, Mich.) charges. The flasks were equipped identically with stirring rods, bushings, condensers, thermocouples/temperature controllers/heating mantles and nitrogen sparges. Then, into each flask, approximately 0.63 g of FASCAT® 4102 (ATOFINA, Philadelphia, Pa.) was added. The reaction mixtures were heated to approximately 215° C. with a slow nitrogen sparge.

Acid value of the reaction mixture in the first (control) flask was monitored periodically. Acid value of the reaction mixture in the second flask, was monitored as well, and approximately 0.63 g of TYZOR TBT (Du Pont, Wilmington, Del.) was added to the second flask after about seven hours of reaction at about 215° C. when the acid value of the reaction mixture fell below approximately 30 mg KOH/g. The reaction in the second flask then continued as with the control. The results of the first (control) flask representing prior art and the second flask representing the presently described technology are shown below:

TABLE I
Dipropylene Glycol-Phthalic Anhydride
Data From Laboratory Studies

| Flask 1 (control) | | Flask 2 | |
|---|---|---|---|
| acid value vs. time | | acid value vs. time | |
| | Acid Value, | | Acid Value, |
| Time, hours | mg KOH/g | Time, hours | mg KOH/g |
| 4.5 | 32 | 6 | 31 |
| 12 | 15.3 | 7 | 26 (add TYZOR ® TBT) |
| 19 | 6.1 | 10 | 9.2 |
| 25 | 2.2 | 13.5 | 2.1 |

Final OH value≈257.5 mg KOH/g Final OH value≈264.5 mg KOH/g

The above comparison shows that the desired acid value of from about 2 to about 2.3 mg KOH/g was achieved in about 13.5 hours utilizing the presently described technology versus about 25 hours for the conventional technology.

Furthermore, continuing the reaction for an additional two hours with the presently described technology achieved a final acid value of about 1.3 mg KOH/g.

### Comparative Example 2

### Preparation Of Polyester Polyos Using Pilot Batches

Production from a pilot facility was designed to emulate the same formulations and conditions as the laboratory batches of Comparative Example 1. The results of a control batch representing prior art and a second batch representing the presently described technology are shown below:

TABLE II
Dipropylene Glycol-Phthalic Anhydride
Data From Pilot Plant Studies

| | Acid value (mg KOH/g) of | |
|---|---|---|
| | | Dual Catalyst |
| Reaction Time | Control Batch | Batch Tin & |
| (hours) | Tin Catalyst Only | Titanium Catalysts |
| 6.5 | 34.4 | — |
| 8 | — | 20.16 (TYZOR ® TBT added) |
| 17 | 19.5 | — |
| 18 | — | 3.28 |
| 20.5 | 10.5 | — |
| 21.5 | — | 2.3 |
| 24 | 8.6 | 1.97 |
| 31 | 5.2 | — |
| 41 | 3.55 | — |
| 45 | 2.4 | — |
| Final OH Value | ~152.7 | ~154.1 |
| After Vacuum | | |
| Stripping | | |
| (mg KOH/g) | | |

The above comparison shows that the desired acid value of from about 2 to about 2.3 mg KOH/g was achieved in about 21.5 hours utilizing the presently described technology versus about 45 hours for the conventional technology.

The products were then “finished” by vacuum stripping at about 180° C. at about 30 inch Hg for about one hour to remove most of the dipropylene glycol monomer. The final acid value of the control batch is about 1.8 mg KOH/g, while the final acid value of the product using the presently described dual catalyst technology reached as low as 0.39 mg KOH/g, which was not previously achieved with dipropylene glycol phthalate utilizing conventional processes.

### Comparative Example 3

### Preparation Of Diethylene Glycol Phthalate Polyester Polyols Using Laboratory Batches

The preparation of laboratory quantities of diethylene glycol phthalate (DEG-Phthalate) polyester polyols used essentially the same equipment and procedure as those used in the laboratory preparation of the polyester polyols as described above in Comparative Example 1. Four DEG-Phthalate polyester polyol samples were prepared in this comparative study. For each sample, approximately 296 grams of phthalic anhydride, approximately 315 grams of diethylene glycol, and approximately 51 grams of soy bean oil were reacted.

In sample one, approximately 0.15 grams of FASCAT® 4102 (ATOFINA, Philadelphia, Pa.) was added first, and then approximately 0.15 grams of TYZOR® TBT (Du Pont, Wilmington, Del.) was added after about four hours of reaction when the acid value of the reaction mixture fell below about 30 mg KOH/g (at about 29.3 mg KOH/g in this example). In sample two, approximately 0.15 grams of FASCAT® 4102 (ATOFINA, Philadelphia, Pa.) and approximately 0.15 grams of TYZOR® TBT (Du Pont, Wilmington, Del.) were added together simultaneously to the initial reaction mixture. In sample three, double amounts of the tin catalyst, i.e., approximately 0.30 grams of FASCAT® 4102 (ATOFINA, Philadelphia, Pa.) were added to the initial reaction mixture. In sample four, double amounts of the titanium catalyst, i.e., approximately 0.30 grams of TYZOR® TBT (Du Pont, Wilmington, Del.) were added to the initial reaction mixture.

The time-acid value data for each of the DEG-phthalate polyester polyol samples are shown in Table III, which show that the DEG-phthalate reaction is faster when dual catalysts were utilized than when double amount of tin catalyst only or titanium catalyst only was utilized. The results also show that when the dual catalysts were added in a staged or sequential manner, the DEG-phthalate reaction proceeded even faster than when the dual catalysts were added simultaneously into the reaction system.

TABLE III
DEG-phthalate Polyester Polyol Data From Laboratory Studies

| | Acid value (mg KOH/g) of PS-2352 from | | | |
|---|---|---|---|---|
| | Dual | Dual | Tin Catalyst | Titanium |
| Reaction | Catalyst | Catalyst | Only | Catalyst Only |
| time | (staged | (simultaneous | (double | (double |
| (hours) | addition) | addition) | amount) | amount) |
| 2 | 52 | 44.8 | — | — |
| 4 | 29.3 | 19.7 | — | 22.4 |
| | (TYZOR ® | | | |
| | TBT added) | | | |
| 4.25 | — | — | 37.3 | — |
| 6 | 15.3 | 16.3 | — | — |
| 7 | — | — | 24.3 | — |
| 8 | — | 2.5 | 20.0 | 15.9 |
| 9 | 2.4 | — | — | — |
| 10 | 0.8 | 1.72 | — | 11.2 |
| 10.5 | — | — | 10.4 | — |
| 11 | 0.3 | — | — | — |
| 13 | — | 0.2 | 2.4 | 2.8 |
| 14.5 | — | — | 1.1 | |
| 16 | — | — | 0.6 | 0.58 |

The present technology is now described in such full, clear, concise and exact terms as to enable any person skilled in the art to which it pertains, to practice the same. It is to be understood that the foregoing describes preferred embodiments of the present technology and that modifications may be made therein without departing from the spirit or scope of the present technology as set forth in the appended claims.

What is claimed is:

1. A process for manufacturing esters comprising:
providing a reaction mixture comprising a carboxyl-containing compound or a derivative thereof and a hydroxyl-containing compound;
charging an effective amount of a first catalyst to the reaction mixture;
heating the reaction mixture; and
charging an effective amount of a second catalyst to the reaction mixture.

2. The process of claim 1, wherein the carboxyl-containing compound or a derivative thereof is a dicarboxylic acid, an anhydride or a lower alkyl (C₁-C₄) ester thereof.

3. The process of claim 1, wherein the hydroxyl-containing compound contains at least two hydroxyl groups.

4. The process of claim 1, wherein the first catalyst is an organotin catalyst.

5. The process of claim 4, wherein the organotin catalyst is used in an amount of from about 50 ppm to about 700 ppm based on the total initial reactant charge weight.

6. The process of claim 1, wherein the second catalyst is an organotitanium catalyst.

7. The process of claim 6, wherein the organotitanium catalyst is used in an amount of from about 50 ppm to about 700 ppm based on the total initial reactant charge weight.

8. The process of claim 1, wherein the second catalyst is charged any time after the first catalyst is charged.

9. The process of claim 1, further comprising additional charging of another effective amount of the second catalyst or a third catalyst to the reaction mixture, and wherein the additional charging can be performed more than once with the same catalyst or a different catalyst.

10. A process for manufacturing esters comprising:
providing a reaction mixture comprising a carboxyl-containing compound or a derivative thereof and a hydroxyl-containing compound;
charging an effective amount of a first catalyst to the reaction mixture;
heating the reaction mixture to progress reaction; and
charging an effective amount of a second catalyst to the reaction mixture when the acid value of the reaction mixture falls below a predetermined acid value.

11. The process of claim 10, wherein the predetermined acid value is in the range of from about 100 mg KOH/g to about 20 mg KOH/g.

12. The process of claim 10, wherein the predetermined acid value is 30 mg KOH/g.

13. The process of claim 10, wherein the carboxyl-containing compound or a derivative thereof is a dicarboxylic acid, an anhydride or a lower alkyl (C₁-C₄) ester thereof.

14. The process of claim 10, wherein the hydroxyl-containing compound contains at least two hydroxyl groups.

15. The process of claim 10, wherein the first catalyst is an organotin catalyst.

16. The process of claim 10, wherein the second catalyst is an organotitanium catalyst.

17. The process of claim 10, further comprising additional charging of another effective amount of the second catalyst or a third catalyst to the reaction mixture, and wherein the additional charging can be performed more than once with the same catalyst or a different catalyst.

18. An ester product produced by a process comprising:
providing a reaction mixture comprising a carboxyl-containing compound or a derivative thereof and a hydroxyl-containing compound;
charging an effective amount of a first catalyst to the reaction mixture;
heating the reaction mixture; and
charging an effective amount of a second catalyst to the reaction mixture.

19. The ester product of claim 18, wherein the second catalyst is charged any time after the first catalyst is charged.

20. The ester product of claim 18, wherein the second catalyst is charged when the acid value of the reaction mixture falls below a predetermined acid value.

21. The ester product of claim 18, wherein the predetermined acid value is in the range of from about 100 mg KOH/g to about 20 mg KOH/g.

22. The ester product of claim 18, wherein the carboxyl-containing compound or a derivative thereof is a dicarboxylic acid, an anhydride or a lower alkyl (C₁-C₄) ester thereof.

23. The ester product of claim 18, wherein the hydroxyl-containing compound contains at least two hydroxyl groups.
